(19) United States
(12) Patent Application Publication
(10) Pub. No.: US 2008/0188799 A1
(43) Pub. Date: Aug. 7, 2008

(54) Medical Reconstitution System with Improved Syringe

(75) Inventors: Andreas Mueller-Beckhaus, Walnut Creek, CA (US); Randy Montellato, San Rafael, CA (US); Martin Vantrieste, Thousand Oaks, CA (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: BAYER HEALTHCARE LLC, Tarrytown, NY (US)

(21) Appl. No.: 11/572,240

(22) PCT Filed: Jul. 22, 2005
(86) PCT No.: PCT/US2005/026298

§ 371 Date: Apr. 16, 2008

Related U.S. Application Data
(60) Provisional application No. 60/606,761, filed on Sep. 2, 2004.

(30) Foreign Application Priority Data
Jul. 24, 2004 (DE) 10 2004 036 051.0

(51) Int. Cl. A61M 5/24; A61M 5/31

(52) U.S. Cl. 604/88; 604/227; 604/201; 604/205

(57) ABSTRACT

The application includes an improved syringe (100) for use with a receptacle of the type having a self-contained movable piercing member. The syringe includes a barrel (114) having a distal end (114d) and a proximal end (114p). At least one finger member (150) is provided proximate to the distal end (114d) of the barrel (114). The finger member (150) is non-slidably attached to the barrel (114) and configured to permit application of a substantial force by one or more fingers of a person's hand such that the syringe (100) is urged in a distal direction.

### FIELD OF THE INVENTION

The invention relates to medical devices and systems, and more particularly relates to an improved injection syringe that is particularly suited for use with a receptacle of the type having a self-contained movable piercing member.

### BACKGROUND

Medical reconstitution devices and systems are known for safely and effectively delivering two-part, reconstituted pharmaceutical compositions for injection into a patient. One embodiment 10 of such a device and system is shown in FIGS. 1 and 2, and is described in detail in U.S. Pat. No. 6,070,623, issued Jun. 6, 2000, and assigned to Biodome America, Inc. (Princeton, N.J.) (hereby incorporated by reference in its entirety). It is marketed under the mark BIO-SET®. As shown in FIG. 1, such a prior art system 10 includes an injection syringe 12 and a receptacle 20. The receptacle 20 includes a vial 22 containing a first constituent 30 in powder or particulate form, for example. The syringe 12 includes a barrel 14 containing a second constituent 40 in a flowable form, such as a liquid diluent, for example. The system 10 is used to mix and recombine the first and second constituents 30, 40 to form a desired pharmaceutical composition 50 for subsequent injection into a patient using the syringe 12.

As shown in FIGS. 1 and 2, the receptacle 20 includes a vial 22 that is closed and sealed by a piercable seal plug or stopper 24 to contain and preserve the first constituent 30 in the vial 22. The seal plug 24 may be formed of a piercable synthetic or rubber material, for example. As shown in FIG. 1, a movable piercing member 25 is positioned above and aligned with the seal plug 24, and includes a centrally positioned piercing member needle 26. The needle 26 is in fluid communication with a first luer-type connector 27 positioned at the upper end of the movable piercing member 25. A sterile shield 28 substantially surrounds the movable piercing member 25, needle, 26, and first connector 27. In FIG. 1, the movable piercing member 25 and needle 26 are shown in a fully-retracted position.

As shown in FIGS. 1 and 2, the syringe 12 has a conventional construction including a barrel 14, a proximal finger brace 16, a distal end member 18, and a plunger 13. The distal end member 18 includes a second connector 19 that is in selective fluid communication with the interior of the barrel 14. The second connector 19 is a luer-type connector configured for mating, sealing engagement with the first connector 27 on the receptacle 20. The second connector 19 also is configured for connection to other compatible medical devices such as an intravenous tube fitting or an injection needle, for example. The syringe barrel 14 typically is constructed of glass, and the proximal finger brace 16 and distal end member 18 typically are constructed of a suitable polymeric material. The syringe 12 is pre-filled with a second flowable constituent 40, such as a diluent, for example.

In use, the second connector 19 on the syringe 12 and the first connector 27 on the receptacle 20 are aligned and sealingly engaged together as shown in FIG. 1. An axial load is applied in a distal direction to the proximal finger brace 16 of the syringe 12 as indicated by arrows F₁ and F₂ in FIG. 1, thereby urging the syringe 12 and movable piercing member 25 in a distal direction toward the seal plug 24 in the receptacle 20. The axial load applied to the proximal finger brace 16 causes the syringe 12 and movable piercing member 25 to move together until the piercing member needle 26 pierces and penetrates the seal plug 24 as shown in FIG. 2. Once the movable piercing member 25 is in a fully-pierced position as shown in FIG. 2, a fluid communication path is established between the vial 22 and syringe barrel 14 through the piercing member needle 26 and the engaged connectors 19, 27. In this fully-pierced position, the flowable second constituent 40 in the syringe 12 can be injected into the vial 22 by advancing the plunger 13 in a distal direction. The flowable second constituent 40 from the syringe 12 mixes with the first constituent 30 in the vial 22 to form a desired reconstituted composition 50. Once thoroughly mixed, the reconstituted composition 50 is drawn into the syringe by drawing the plunger 13 in a proximal direction. The syringe 12 is then ready for use with a compatible injection needle (not shown) to inject the reconstituted medical composition 50 into a patient.

Though a medical reconstitution system 10 like that described above provides a convenient and effective system for reconstituting first and second constituent ingredients into a medical composition for injection into a patient, such a system 10 has at least one shortcoming. In some cases, a transverse or eccentric load (such as that indicated by arrow F₃ in FIG. 1) may inadvertently be applied at or near the proximal end of the syringe 12 as the syringe 12 is used to push the movable piercing member 25 toward the receptacle 20. Due to the length of the syringe 12, such an eccentric load F₃ may create a substantial bending moment at the distal end of the syringe at the engaged connectors 19, 27. Such an eccentric load “F₃” also may create a substantial bending moment in the syringe barrel 14. If the resultant bending loads are sufficiently large, at least a portion of the system 10 may be distorted, damaged, or broken. In a severe case, the syringe tip 19 or glass barrel 14 may break under the resultant bending stresses, thereby subjecting a user to a potentially severe safety hazard.

Accordingly, there is a need for an improved medical reconstitution system that includes the advantages of known systems like that described above, but is less susceptible to damage or breakage during use than known medical reconstitution systems.

### SUMMARY

The invention includes a syringe for hypodermic, intravenous, or intramuscular injection. The syringe includes a barrel having a distal end and a proximal end. At least one finger member is provided proximate to the distal end of the barrel. The finger member is positively attached to the barrel and is configured to permit application of a substantial force by one or more fingers of a person's hand such that the syringe is urged in a distal direction. In the present specification and the appended claims, the term “distal” refers to a position or direction that is toward the injection end of a syringe, and the term “proximal” is used to refer to a position or direction that is distant from the injection end of a syringe.

The invention also includes a medical reconstitution system for a composition comprising first and second mixable constituents. The system includes a receptacle containing the first constituent. The receptacle includes a container having a piercable seal. A piercing member is configured to selectively pierce the piercable seal. The piercing member is operable between a retracted position and a fully-pierced position. The receptacle further includes a first connector connected to and in fluid communication with the piercing member, and a sterile shield substantially surrounding the seal and piercing member. The system also includes a syringe containing the second constituent. The syringe includes a barrel having a distal end, a proximal end, and a second connector on the distal end. The second connector is configured to engage the first connector of the receptacle and to provide a substantially fluid-tight connection therewith. At least one finger member is provided proximate to the distal end of the barrel. The finger member is configured to permit application of a force by one or more fingers of a person's hand such that the syringe is urged in a distal direction. When the piercing member is in the retracted position and the first and second connectors are sealingly engaged together, the piercing member is capable of being advanced to the fully-pierced position by pushing and displacing the finger member in the distal direction toward the receptacle.

The invention further includes a method for preparing a two-constituent composition for injection. The method includes providing a first constituent in a receptacle having a piercable seal, and having a piercing member movable between a retracted position and a fully-pierced position. The receptacle also includes a first connector in fluid communication with the piercing member. The method also includes providing a second constituent in an injection syringe having at least one finger member proximate to a distal end of the syringe. The syringe also includes a second connector on the distal end of the syringe. The method next includes connecting the first and second connectors while the piercing member is in the retracted position, and displacing the piercing member from the retracted position to the fully-pierced position by pushing the distal finger member toward the receptacle, thereby causing the piercing member to pierce the seal. In addition, the method includes injecting the second constituent from the syringe into the receptacle through the first and second connectors and piercing member, and causing the first and second constituents to mix together in the receptacle to form the composition. Lastly, the method includes drawing the composition into the syringe from the receptacle through the piercing member and the first and second connectors.

A reading of the following detailed description together with the drawings will provide a more thorough understanding of the invention.

### BRIEF DESCRIPTION OF THE DRAWINGS

FIG. 1 is a longitudinal cross sectional view of a prior art medical reconstitution device;

FIG. 2 is another longitudinal cross section of the prior art medical reconstitution device of FIG. 1 (shown as after activation of the piercing member);

FIG. 3 is a perspective view of an embodiment of an injection syringe according to the invention;

FIG. 4 is a longitudinal cross sectional view of the syringe of FIG. 3, including a partially removable sealing cap;

FIG. 5 is a longitudinal cross sectional view of the syringe of FIG. 4 with a removable portion of the sealing cap removed;

FIG. 6 is a top perspective view of one embodiment of a distal finger member for a syringe according to the invention;

FIG. 7 is a bottom perspective view of the distal finger member of FIG. 6;

FIG. 8 is a plan view showing the top of the distal finger member shown in FIGS. 6 and 7;

FIG. 9 is a cross sectional view of the distal finger member shown in FIGS. 6-8 taken along line 9-9 in FIG. 8;

FIG. 10 is a plan view showing the bottom of the distal finger member shown in FIGS. 6-9;

FIG. 11 is a cross sectional view of the distal finger member shown in FIGS. 6-10 taken along line 11-11 in FIG. 10;

FIG. 12 is a longitudinal cross section of a medical reconstitution system according to the invention with its movable piercing member in a fully retracted position; and

FIG. 13 is a longitudinal cross section of the medical reconstitution system of FIG. 12 with the movable piercing member in a fully pierced position.

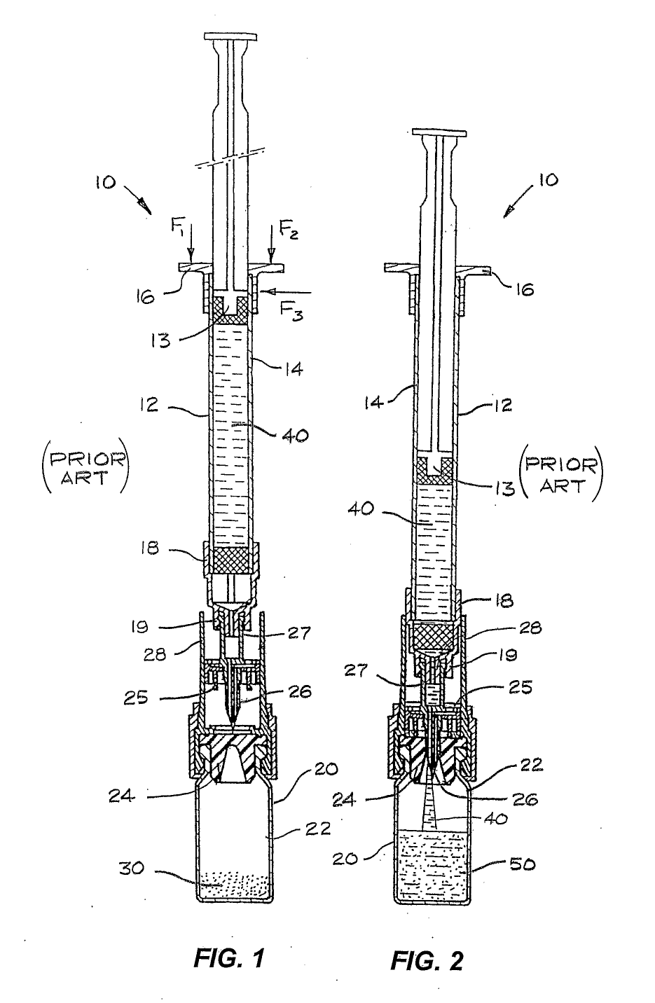

### DETAILED DESCRIPTION

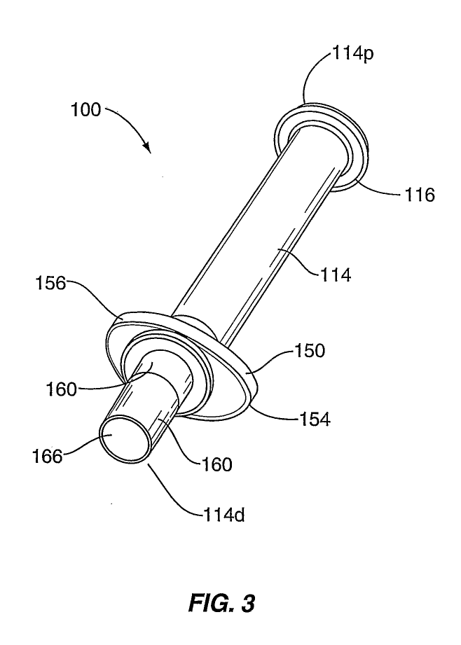

An improved injection syringe 100 according to the invention is shown in FIGS. 3-5. As shown in FIG. 3, the syringe includes a barrel 114, a proximal finger brace 116, and a distal end member or sealing cap 160 having a first connector 162. In the following description, the term “distal” is used to refer to a position or direction that is toward the injection end of the syringe 100, and the term “proximal” is used to refer to a position or direction that is distant from the injection or distal end of the syringe 100. The syringe 100 further includes a distal finger member or distal finger brace 150 that is positioned proximate to the distal end of the syringe 100. In other words, the distal finger member or brace 150 is positioned on the syringe barrel 114 such that the brace 150 is nearer the distal end 114d of the syringe barrel 114 than the opposed proximal end 114p of the barrel 114. The distal finger member 150 is configured to permit an axial force to be applied to the syringe 100 in a distal direction with one or more fingers of a person's hand or hands. In the embodiment 100 shown in FIG. 3, the distal finger member 150 includes a first outwardly protruding tab portion 154, and an opposed second outwardly protruding tab portion 156.

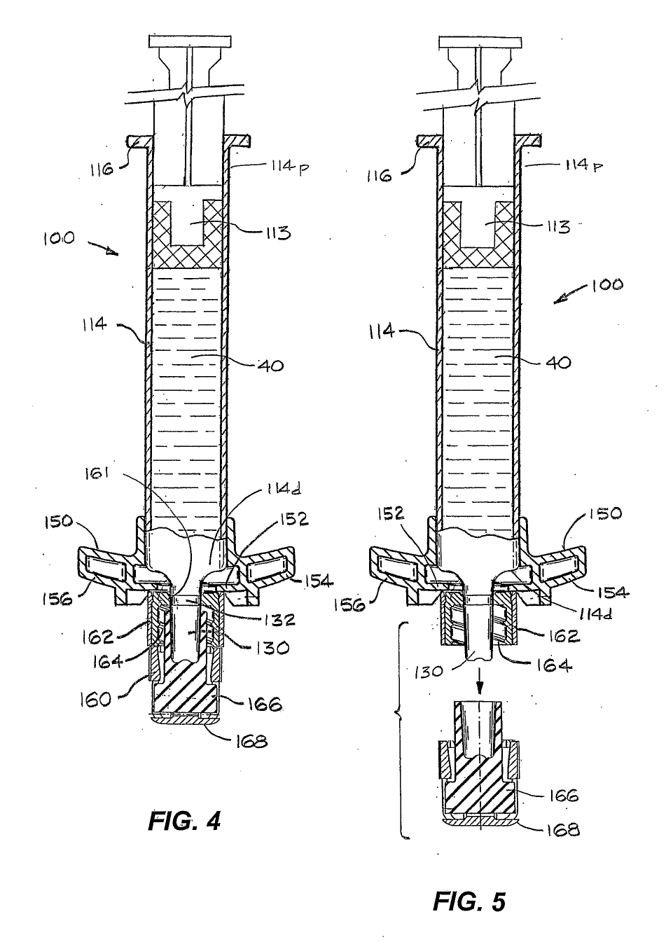

Referring to FIGS. 4 and 5, the syringe 100 includes an elongated barrel 114 having a plunger 113 disposed therein. The distal end of the barrel includes a tapered nipple 130 having a notch or groove 132 therearound. In a prefilled syringe 100, a flowable constituent 40 is disposed within the barrel 114. In order to seal and protect the flowable constituent 40 within the syringe 100, a tamper-evident sealing cap 160 may be secured on the syringe nipple 130 by the groove 132 as shown. One such sealing cap 160 that may be used with a syringe 100 according to the invention is a compatible V-OVS® closure available from Vetter Pharma-Turm Inc., for example. As shown in FIGS. 4 and 5, the sealing cap 160 includes a retained cap-shaped portion 162 and a removable sealing portion 166. The retained cap-shaped portion 162 includes a collar portion 161 that is received in the groove 132, thereby positively retaining the cap-shaped portion 162 on the nipple 130 and barrel 114. The sealing portion 166 includes a resilient sealing member 166 that mates with and seals the open tip of the syringe nipple 130 when the sealing cap 160 is unopened, as shown in FIG. 4. When the sealing portion 166 of the sealing cap 160 is removed from the retained portion 162 as shown in FIG. 5, the syringe nipple 130 is unsealed, and capable of dispensing the flowable constituent 40 from the syringe 100.

As shown in FIG. 5, the exposed nipple 130 and positively retained portion 162 of the sealing cap 160 combine to form a male luer-type connector as is known in the art. The tapered nipple 130 is sized and shaped to be received by a compatible female luer connector on a vial or other medical device. Threads 164 in the retained portion 162 of the cap 160 are configured to lockingly engage mating threads on a compatible female luer-type connector, thereby forming a leak-tight seal between the connectors.

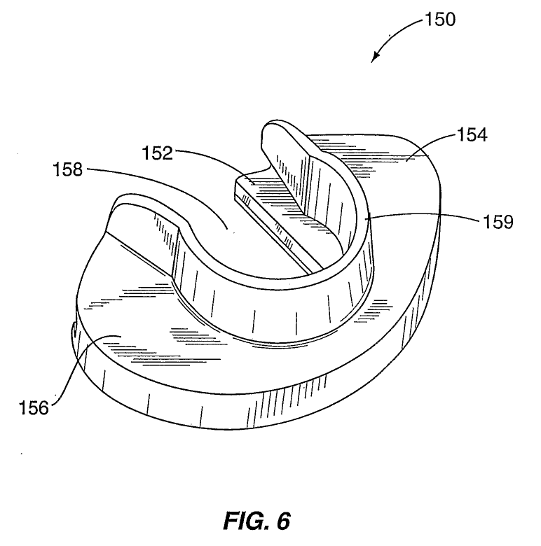

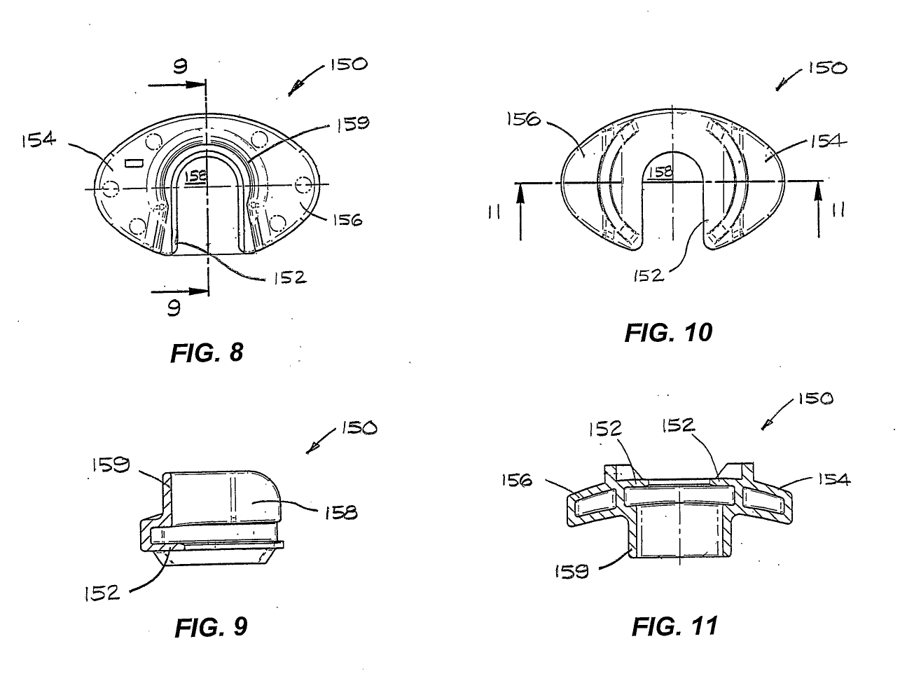

One embodiment of a distal finger member 150 for use with a syringe 100 according to the invention is shown in FIGS. 4-11. In this embodiment, the distal finger member 150 is configured to securely snap onto the distal end of a pre-filled syringe 100 as shown in FIGS. 3-5. The distal finger member 150 includes a first tab portion 154 and an opposed second tab portion 156. The tab portions 154, 156 each are configured to receive at least one fingertip to apply a distally-directed force to the syringe 100. As shown in FIGS. 6-8 and 10, the distal finger member 150 includes a substantially U-shaped recess 158 including a central bore for receiving at least a portion of the distal end 114d of the syringe barrel 114 therein. A substantially cylindrical wall 159 at least partially surrounds a portion of the recess 158. As shown in FIGS. 6-9 and 11, the wall 159 preferably includes an inner bore having a diameter that is equal to or slightly less than the major diameter of a mating syringe barrel 114. When engaged on a syringe barrel 114, the cylindrical wall 159 grips the barrel to align and at least partially secure the distal finger member 150 on the barrel 114.

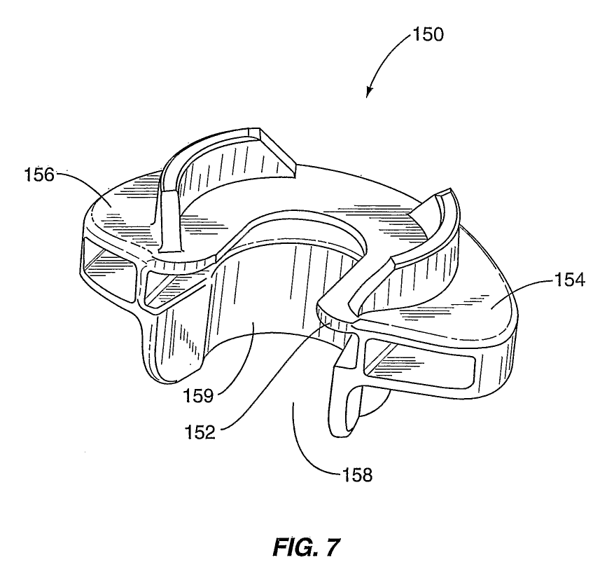

As shown in FIGS. 7-11, a substantially U-shaped lip 152 also surrounds the U-shaped recess 158 and is substantially coextensive therewith. The opposed edges of the U-shaped lip 152 are spaced apart by a distance that is smaller than the major diameter of a mating syringe barrel 114, and that is at least slightly larger than the largest diameter of the nipple 130 on the mating syringe barrel 114. As shown in FIGS. 4 and 5, the thickness of the lip 152 is selected such that the lip 152 is capable of being received in a space or gap between the distal end 114d of the syringe barrel 114 and the retained portion 162 of the sealing cap 160. The lip 152 functions to locate the distal finger member 150 on the distal end 114d of a syringe 100, to substantially prevent the finger member 150 from sliding in either a distal or proximal direction on the barrel 114, and to transfer a substantial distally-directed force from the distal finger member 150 to the syringe nipple 130 through the engaged connector 162. The positive engagement between the collar 161 of the connector 162 and the groove 132 in the nipple 130 prevents the connector 162 from disengaging from the nipple 130 as a substantial distally-directed force is applied to the distal finger member 150 and syringe 100.

Preferably, the distal finger member 150 is constructed of a resilient material that permits the U-shaped member 150 to elastically deform and recover as the member 150 is snapped onto the distal end 114d of a syringe barrel 114. For example, the distal finger member 150 may be formed of molded polypropylene or another suitable polymeric material.

The distal finger member 150 may have any suitable configuration that permits a substantial axial force to be applied to the distal end of a syringe in a distal direction at or near the distal end of the syringe with one or more fingers of a person's hand or hands. For example, the distal finger member 150 may include an outwardly protruding disc that substantially surrounds and positively engages the distal end of the syringe barrel, or any other suitable shape.

In a preferred embodiment of the syringe 100, the barrel 114 is a substantially transparent glass tube that permits a substantially unobstructed view of the syringe's contents 40. Alternatively, the barrel 114 may be constructed of a suitable polymeric material. The proximal finger brace 116 and plunger 113 may be constructed of any suitable material, such as a suitable polymeric material or the like, as is known in the art.

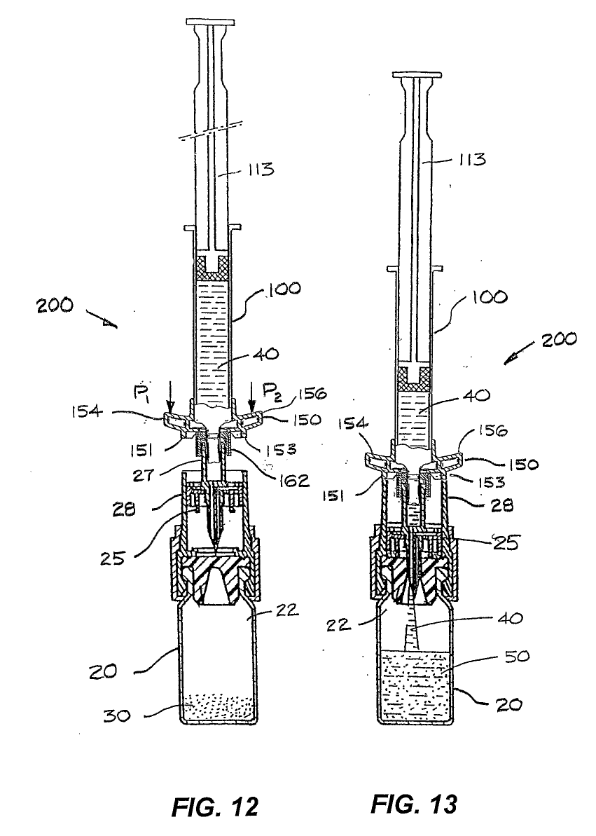

The syringe 100 described above is particularly well suited for use with a receptacle 20 having a movable piercing member 25 like that described above and shown in FIGS. 12 and 13. In order to reconstitute a powdered or freeze-dried first constituent 30 in a vial 22 with a flowable second constituent 40 in a syringe 100, the syringe 100 is aligned with the receptacle 20 and the first and second connectors 162, 27 are sealably engaged together. In practice, a person may support the receptacle 20 with one hand (such as on a flat, horizontal surface), while applying force or pressure to the distal finger member 150 in a distal direction with one or more fingers of the opposite hand. The distal direction of the force or pressure applied to the distal finger member 150 is indicated in FIG. 12 by arrows P₁ and P₂.

As this distally-directed force or pressure is applied to the distal finger member 150 of the syringe 100, the movable piercing member 25 of the receptacle 20 is pushed downward until the movable piercing member 25 is in a fully-pierced position as shown in FIG. 13. In this fully-pierced position, a fluid communication path is established between the barrel 114 of the syringe 100, and the vial 22 of the receptacle 20. In one embodiment of the system 200 as shown in FIG. 13, at least a portion of the distal finger member 150 meets or contacts at least a portion of the receptacle 20 when the movable piercing member 25 reaches the fully-pierced position. For example, in the embodiment shown in FIG. 13, the stops 151, 153 on the bottom of the distal finger member 150 of the syringe 100 contact the upper edge of the sterile shield 28 on the receptacle 20, thereby providing a positive indication to a user that the hidden movable piercing member 25 is in the fully-pierced, activated position.

Because the distally-directed force or pressure is applied to the distal finger member 150 at a location that is near the distal end of the syringe 100, even in instances where an eccentric or transverse load inadvertently is applied to the syringe in this distal location, any resultant bending moment on the engaged connectors 162, 27 or syringe barrel 114 is small compared to the bending moment that would result if the same transverse load was applied at or near the proximal end of the syringe 100. Accordingly, the improved syringe 100 and medical reconstitution system 200 provides a substantially lower risk of damage or breakage than known medical reconstitution devices and methods like the prior art device and method described above.

The improved syringe 100 and improved medical reconstitution system 200 described above is particularly important to frequent users of such systems, such as patients with hemophilia. Such persons must regularly inject themselves with an antihemophilic factor in order to improve blood coagulation. Such persons typically self-administer such injections at home. One such antihemophilic factor is recombinant KOGENATE® FS, produced by Bayer HealthCare LLC. Such a two-part recombinant hemophilic factor typically includes a dried active constituent and a compatible diluent. While prior art medical reconstitution systems like the system 10 described above provide a needleless system and method for reconstituting a recombinant antihemophilic factor such as KOGENATE® FS, the improved system 200 and method described above provides a system having all of the advantages of the prior system 10, plus the further advantage of a reduced likelihood of accidental damage or breakage of the syringe 100 or receptacle 20 during reconstitution. Certain embodiments of the improved system 200 can also provide a clear visual indication to a user when the system 200 has been fully activated, e.g. when stops 151 and 153 are in full or close contact with a component of a reconstitution system, e.g. such as sterile shield 28 (see FIG. 13). Accordingly, the invention provides a reconstitution process that is simple, convenient, and safer than existing systems.

The various embodiments of the invention described above are provided for the purpose of illustrating various features and aspects of the invention, and are not intended to limit the scope of the invention. Persons of ordinary skill in the art will recognized that certain modifications may be made to the described embodiments without departing from the scope of the present invention. For example, though the embodiment of a syringe 100 is described above as having a distal finger member 150 that is separate and removable from its mating syringe barrel 114, the finger member and syringe barrel may be integrally formed together of a suitable material. Alternatively, separate protruding finger tabs may be attached to an outer wall of the syringe barrel with a suitable adhesive or the like. All such modifications are intended to be within the scope of the appended claims.

What is claimed is:

1. An injection syringe comprising:
(a) a barrel having a distal end and a proximal end; and
(b) at least one finger member non-slidably attached to a distal portion of the barrel, the finger member being configured to permit application of a substantial force by one or more fingers of a person's hand such that the syringe is urged in a distal direction.

2. A syringe according to claim 1 wherein the finger member comprises:
(a) a first finger tab on a first side of the barrel; and
(b) a second finger tab on a second side of the barrel.

3. A syringe according to claim 2 wherein the first and second sides of the barrel are substantially opposite each other.

4. A syringe according to claim 1 wherein the barrel comprises glass.

5. A syringe according to claim 1 wherein the finger member comprises a polymeric material.

6. A syringe according to claim 1, further comprising a first connector on the distal end of the barrel, the first connector being configured to matingly engage a second connector on a medical device.

7. A syringe according to claim 6 wherein the first connector is configured to matingly engage a second connector on a composition receptacle.

8. A syringe according to claim 1, further comprising a pharmaceutical composition within the barrel.

9. A syringe according to claim 1, wherein the finger member is removably attached to the distal portion of the barrel.

10. A medical reconstitution system for a composition comprising first and second mixable constituents, the system comprising:
(a) a receptacle containing the first constituent, the receptacle comprising:
(i) a container including a piercable seal;
(ii) a piercing member configured to selectively pierce the piercable seal, the piercing member being operable between a retracted position and a fully-pierced position;
(iii) a first connector connected to and in fluid communication with the piercing member; and
(iv) a sterile shield substantially surrounding the seal and piercing member and
(b) a syringe containing the second constituent, the syringe comprising:
(i) a barrel having a distal end, a proximal end, and a second connector on the distal end, the second connector being configured to engage the first connector of the receptacle and provide a substantially fluid-tight connection therewith;
(ii) at least one finger member non-slidably attached to a distal portion of the barrel, the finger member being configured to permit application of a force by one or more fingers of a person's hand such that the syringe is urged in a distal direction;
(c) wherein when the piercing member is in the retracted position and the first and second connectors are sealingly engaged together, the piercing member is capable of being advanced to the fully-pierced position by pushing and displacing the finger member in the distal direction toward the receptacle.

11. A system according to claim 10 wherein the first constituent comprises an antihemophilic factor and the second constituent comprises a diluent therefor.

12. A system according to claim 10, further comprising an injection needle configured to sealingly connect to the second connector of the syringe.

13. A system according to claim 10 wherein at least a portion of the finger member of the syringe contacts a portion of the sterile shield when the piercing member is advanced to the fully pierced position.

14. A system according to claim 10 wherein the barrel of the syringe comprises glass.

15. A system according to claim 10 wherein the syringe further comprises a plunger disposed in the barrel.

16. A system according to claim 10 wherein the first and second connectors are compatible luer-lock connectors.

17. A method of delivering a multi-constituent composition for injection, the method comprising:
(a) providing a first constituent in a receptacle having a piercable seal, a piercing member movable between a retracted position and a fully-pierced position, and a first connector in fluid communication with the piercing member;
(b) providing a second constituent in an injection syringe having at least one finger member proximate to a distal end of the syringe, and having a second connector on the distal end of the syringe;
(c) connecting the first and second connectors while the piercing member is in the retracted position;
(d) displacing the piercing member from the retracted position to the fully-pierced position by pushing the distal finger member toward the receptacle, thereby causing the piercing member to pierce the seal;
(e) injecting the second constituent from the syringe into the receptacle through the first and second connectors and piercing member;
(f) causing the first and second constituents to mix together in the receptacle to form the composition; and
(g) drawing the composition into the syringe from the receptacle through the piercing member and through the first and second connectors.

18. The method according to claim 17 wherein at least a portion of the finger member of the syringe meets a portion of the receptacle when the piercing member is in the fully-pierced position to provide a visual indication to a user that the syringe is fully engaged with the receptacle.

19. The method according to claim 17 wherein the syringe and receptacle are substantially transparent such as to substantially permit viewing of the first and second constituents and mixtures thereof within the receptacle and syringe.

20. The method according to claim 17 wherein the first constituent is an antihemophilic factor and the second constituent is a compatible diluent therefor.

21. A distal finger brace for a hypodermic syringe having a barrel with a proximal end and a distal end, the distal finger brace comprising:
(a) a central bore sized and configured to receive at least a portion of the syringe barrel proximate to the distal end of the barrel;
(b) a lip configured to positively engage the distal end of the barrel; and
(b) at least one outwardly extending finger tab.

22. A distal finger brace according to claim 21, further comprising a U-shaped recess that permits the brace to be engaged onto the syringe barrel from a side of the barrel.

23. A distal finger brace according to claim 21, further comprising at least one upwardly extending wall that at least partially surrounds the central bore.

24. A distal finger brace according to claim 21, further comprising at least one downwardly extending stop.

25. A distal finger brace according to claim 21, further comprising a resilient polymeric material.
